(19) United States
(12) Patent Application Publication
(10) Pub. No.: US 2004/0265846 A1
(43) Pub. Date: Dec. 30, 2004

(54) Adrenergic receptors

(75) Inventor: Debra A. Schwinn, Durham, NC (US)

Correspondence Address: NIXON & VANDERHYE, PC, 1100 N GLEBE ROAD, 8TH FLOOR, ARLINGTON, VA, 22201-4714, US

(73) Assignee: DUKE UNIVERSITY, Durham, NC

(21) Appl. No.: 10/715,844

(22) Filed: Nov. 19, 2003

Related U.S. Application Data
(60) Provisional application No. 60/427,219, filed on Nov. 19, 2002.

(51) Int. Cl. C12Q 1/68

(52) U.S. Cl. 435/6

(57) ABSTRACT

The present invention relates, in general, to adrenergic receptors and, in particular, to α₁ₐ-adrenergic receptors and variants thereof The invention further relates to methods of using such variants in disease diagnosis and drug development.

This application claims priority from U.S. Provisional Application No. 60/427,219, filed Nov. 19, 2002, the entire content of that application being incorporated herein by reference.

### TECHNICAL FIELD

The present invention relates, in general, to adrenergic receptors and, in particular, to α₁ₐ-adrenergic receptors and variants thereof. The invention further relates to methods of using such variants in disease diagnosis and drug development.

### BACKGROUND

α₁-Adrenergic receptors (α₁ARs) belong to a superfamily of G protein-coupled receptors (GPCRs) that share a common overall structure with 7 hydrophobic transmembrane (TM) helices. α₁ARs are activated by endogenous catecholamines norepinephrine (NE) and epinephrine, thereby mediating actions of the sympathetic nervous system. Stimulation of α₁ARs predominantly activates the Gq/11 protein, resulting in hydrolysis of membrane phospholipids via phospholipase Cβ; resultant second messengers include inositol triphosphate (IP₃) and diacylglycerol, which mobilize intracellular calcium and activate protein kinase C, respectively (reviewed in Graham et al, Circ. Res. 78:737-749 (1996), Michelotti et al, Pharmacol. Ther. 88:281-309 (2000)).

Three α₁AR subtypes have been identified based on results from pharmacological and molecular cloning studies—α₁ₐ, α_{1b} and α_{1d}. These subtypes are present in a wide variety of organs and tissues including human brain, liver, prostate, vascular smooth muscle, and myocardium (Price et al, Mol. Pharmacol. 45:171-175 (1994), Rudner et al, Circulation 100:2336-2343 (1999)). α₁ₐARs have been shown to play important roles in the dynamic component of benign prostatic hyperplasia (Price et al, J. Urol. 150:546-551 (1993), Forray et al, Mol. Pharmacol. 45:703-708 (1994), Marshall et al, Br. J. Pharmacol. 115:781-786 (1995)) and in the development of myocardial hypertrophy (Rokosh et al, J. Biol. Chem. 271:5839-5843 (1996), Autelitano and Woodcock, J. Mol. Cell Cardiol. 30:1515-1523 (1998)). Previous studies (Rudner et al, Circulation 100:2336-2343 (1999)) have demonstrated that the α₁ₐAR subtype predominates in human resistance vessels which mediate sympathetically-derived vasoconstriction. The α₁ₐAR is also located on human chromosome 8p21-p22, in a region previously associated with blood pressure regulation (Wu et al, J. Clin. Invest. 97:2111-2118 (1996)). Taken together these findings suggest that human α₁ₐARs contribute to blood pressure homeostasis and potentially the pathogenesis of diseases such as hypertension.

In addition to tissue specific differences in both AR subtype distribution and expression levels, naturally occurring human receptor polymorphisms have been shown to modulate sympathetically-mediated physiologic responses. Most data in this regard originate from βARs (β₁, β₂ and β₃) and α₂ARS (α₂ₐ, α_{2b}, α_{2c}) (Ranade et al, Am. J. Hum. Genet. 70:935-942 (2002), Svetkey et al, Hypertension 27:1210-1215 (1996), Erickson and Graves, Drug. Metab. Dispos. 29:557-561 (2001), Rosmond et al, J. Intern. Med. 251:252-257 (2002), Small et al, J. Biol. Chem. 276:4917-4922 (2001), Snapir et al, J. Am. Coll. Cardiol. 37:1516-1522 (2001), Buscher et al, Trends Pharmacol. Sci. 20:94-99 (1999)). More limited genetic variant studies have been performed within the α₁AR family, with rare, non-functional, truncated α₁ₐARs resulting from incomplete splicing of the two exons (Coge et al, Biochem. J. 343(Pt. 1):231-239 (1999)). The only polymorphic site in the full-length human α₁ₐAR functionally analyzed to date, R492C, is located in the carboxyl terminal portion of the receptor and was discovered via a PstI restriction fragment length polymorphism (RFLP) (Hoehe et al, Hum. Mol. Genet. 1:349 (1992)). Since this polymorphism had no effect on receptor behavior, it is not surprising that no association has been shown for this variant for some diseases (benign prostatic hyperplasia (Shibata et al, Br. J. Pharmacol. 118:1403-1408 (1996)), depression (Bolonna et al, Neurosci. Lett. 280:65-68 (2000)), or essential hypertension (Xie et al, Pharmagenetics 9:651-656 (1999)).

The present invention results, at least in part, from studies designed to define naturally occurring polymorphisms for the human α₁ₐAR and the functional significance of same. These studies have resulted in the identification of α₁ₐAR variants and have made possible methods of disease diagnosis and treatment as well as drug screening.

### SUMMARY OF THE INVENTION

The present invention relates generally to adrenergic receptors. More specifically, the invention relates to α₁ₐAR and variants thereof and to methods of using such variants in disease diagnosis and drug development. The invention further relates to methods of treating diseases associated with α₁ₐAR variants.

Objects and advantages of the present invention will be clear from the description that follows.

### BRIEF DESCRIPTION OF THE DRAWINGS

FIG. 1. Approximate location of SNPs in gene.

FIG. 2. Seven-transmembrane-spanning model of human α₁ₐAR showing the primary amino acid sequence. Key residues are colored including SNP sites (yellow, with amino acid number listed next to SNP), and important residues for agonist binding (red) and antagonist binding (green). The salt bridge is formed between D106 (red and blue, also identified as an important residue in agonist binding) in TM3 and K309 (blue) in TM7. This model was based on the results of several mutagenesis studies (Hwa et al, J. Biol. Chem. 270:23189-23195 (1995), Hwa et al, J. Biol. Chem. 271:6322-6327 (1996), Porter et al, J. Biol. Chem. 271:28318-28323 (1996), Waugh et al, J. Biol. Chem. 275:11698-11705 (2000), Waugh et al, J. Biol. Chem. 276:α125366-25371 (2001), Zhao et al, Mol. Pharmacol. 50:118-1126 (1996), Chen et al, J. Biol. Chem. 274:16320-16330 (1999), Hamaguchi et al, Biochemistry 35:14312-14317 (1996)).

FIGS. 3A-3C. Effect of human α₁ₐAR SNPs on IP signaling in high-expression stable clones (receptor densities 1.53-2.37 pmol/mg protein). Cells labeled with [³H]inositol were treated for 20 min with increasing concentrations (10⁻⁸-10⁻⁴M) of NE. For each construct, binding assays, IP assays, and cell count determinations were performed in parallel plates. FIG. 3A. Basal IP release (without agonist stimulation). FIG. 3B. Maximal IP release in response to NE, normalized for receptor/well. FIG. 3C. EC₅₀ values for NE stimulation of IP production. **P<0.01 compared with α₁ₐAR WT. n=3-8.

FIGS. 4A-4C. Effect of human α₁ₐAR SNPs on IP signaling in low-expression stable clones (receptor densities 0.21-0.44 pmol/mg protein). Cells labeled with [³H]inositol were treated for 20 min with various concentrations (10⁻⁸-10⁻⁴M) of NE. For each construct, IP assay, binding assay and cell number were measured in paralleled plates. FIG. 4A. Basal IP release. FIG. 4B. Maximal IP release in response to NE, normalized for receptor/well. FIG. 4C. EC₅₀ values for NE stimulation of IP production. **P<0.01 compared with α₁ₐAR WT. n=3-6.

FIGS. 5A-5C. Effect of human α₁ₐAR SNPs on IP signaling in transiently transfected rat-1 fibroblasts (receptor densities 1.30-1.98 pmol/mg protein). Cells labeled with [³H]inositol were treated for 20 min with various concentration (10⁻⁸-10⁻⁴M) of NE. For each construct, IP assay, binding assay and cell number were measured in paralleled plates. FIG. 5A. Basal IP release. FIG. 5B. Maximal IP release in response to NE, normalized for receptor/well. FIG. 5C. EC₅₀ values for NE stimulation of IP production. **P<0.01 compared with α₁ₐAR WT. n=4.

FIGS. 6A and 6B. Effects of human α₁ₐAR SNPs on norepinephrine (NE)-induced desensitization. [³H]inositol-labeled rat-1 fibroblasts stably expressing wild type (WT) α₁ₐAR or its SNPs were pretreated for 10 min with 10⁻⁵ M NE, washed, then incubated with 10⁻⁴ M NE for 20 min in the presence of LiCl, and total IPs were quantitated. The extent of desensitization was expressed as percent of reduction of IP response in pretreated cells compared with the response in naive cells. n=3-6. FIG. 6A. High-expression clones. FIG. 6B. Low-expression clones.

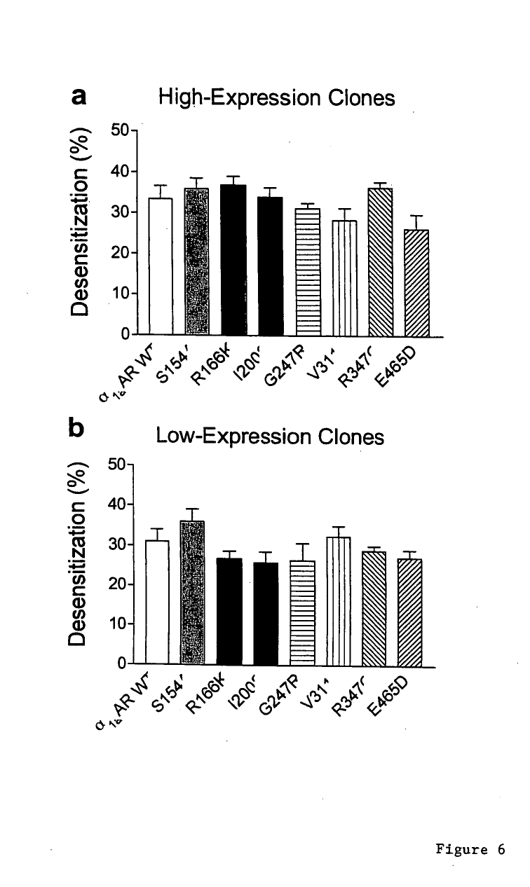

FIG. 7. Surface receptors of SNP G247R and wild type α₁ₐAR and norepinephrine-induced internalization. Low-expressing SNP G247R and WT receptor cells grown in 6-well plates were treated with 10 μM NE or vehicle for 1 h and then fixed with 3.7% formaldehyde for 10 min. Rat monoclonal anti-HA antibody 3F10-peroxidase (1:1000 dilution) or 3F10 (1:1000 dilution, for testing background signal) was incubated with fixed cells for 30 min. Following one wash with block buffer and twice washes with PBS, cells were incubated with ABTS solution (Boehringer Mannheim) for 1 h. 1 ml of solution was transferred from each well to 48-well plates and O.D. values were read at 405 nm. The specific O.D. value is from total O.D. subtracting by background signal. n=3.

FIGS. 8A and 8B. SNP G247R stimulates cell growth. FIG. 8A. SNP G247R stimulates cell proliferation. Low-expression clones were planted at same density. After 48 h incubation in complete medium, cells were trypsinized and counted. FIG. 8B. Cells were planted in 12-well plates at 5×10⁴ cells/well. After washed once with PBS gently, cells were harvested by 250 μl of lysis buffer (1% nonidet P-40 and 0.5% sodium deoxycholate) at 4, 24, 48, 72 h points. 50 μl of samples were used for total protein measurement by BCA protein assay reagent kit (Pierce).

### DETAILED DESCRIPTION OF THE INVENTION

The present invention results, at least in part, from studies designed to identify naturally occurring genetic variants of the human α₁ₐ-adrenergic receptor (α₁ₐAR; previously named α_{1c}AR—for review on nomenclature see Hieble J P et al, Pharmacol, Rev. 47:267-270 (1995)) and to determine whether such variants are associated with human diseases such as hypertension, benign prostatic hypertrophy, or other diseases. In order to examine the biologic effects of α₁ₐAR genetic variants, a clinical measure was selected that was known to be affected by this receptor system—vasoconstriction (which can be measured in terms of blood pressure). In order to address the question of whether α₁ₐAR genetic variants are associated with altered blood pressure, DNA matched with highly phenotyped patient data for blood pressure and other medical diseases/drugs was obtained. There were 4 patient sets, as well as the n=90 Coriell SNP discovery panel, a publicly available resource of genomic DNAs enriched for minorities (a group of individuals that has been shown over the years to have higher overall genetic variability).

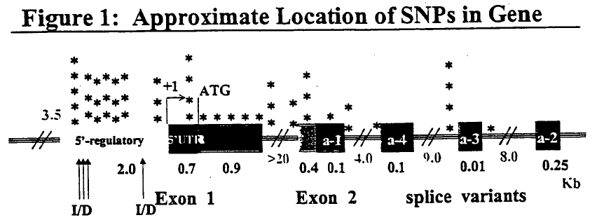

DNA was sequenced from individuals at the highest and lowest blood pressures (5000 bp DNA from n=281 total individuals was sequenced using rapid throughput DNA sequencers). As a result, 49 genetic variants of the human α₁ₐAR have been identified over the 5 kb of sequence investigated, 46 of which are single nucleotide polymorphisms (SNPs), 1 insertion/deletion, and 3 microsatellite repeats. The exact location of these genetic variants is shown in Table 1 (the α₁ₐAR gene has 2 major exons and 3 additional splice variants in addition to the originally described wild type sequence (Table 2-1^{st} exon with its 5′-regulatory/UTR and 3′ intron sequence, Table 3-2^{nd} exon with associated 5′ intron and 3′ sequences (splice variant a-1 is part of exon 2 sequence), Table 4—splice variant a-4 and its associated 5′ and 3′ nucleotides, Table 5—splice variant a-3 with its associated 5′/3′ sequences (see also schematic is shown in FIG. 1). As detailed in the Example below, cDNAs expressing the coding region SNPs that alter amino acid sequence have been prepared and characterized for biologic function; biologic effects in terms of ligand binding to agonists or antagonists are described, as is the association of one coding region SNP with altered cell growth. (Two SNPs have been reported in dbSNP—A6804 (I200S, released on Aug. 15, 2001) and A6944 (G247R, released on Aug. 28, 2002)—there is no reference to any function, confirmation, or association with disease for either of these SNPs.)

Thus, the invention relates to α₁ₐAR variants and to methods of detecting same. These variants can be associated with diseases that involve the sympathetic nervous system and can be used as screening tools to predict/monitor onset, severity or treatment of diseases including prostate disease (benign prostatic hyperplasia or prostate cancer), cardiovascular disease, psychiatric disease, hypotensive syndromes, cancer, etc. At least one variant of the invention, A6944 (G247R at the amino acid level) can be expected to be associated with cancer and coronary artery disease.

The expression of an α₁ₐAR variant in an individual can be detected at either the DNA or RNA level using any of a variety of techniques well known in the art (including microarray-and “gene chip”-based technologies). Examples of such techniques include restriction-fragment-length-polymorphism detection based on allele-specific restriction-endonuclease cleavage (Kan et al, Lancet ii:910-912 (1978)), hybridization with specific oligonucleotide probes (Wallace et al, Nucl. Acids Res. 6:3543-3557 (1978)), including immobilized oligonucleotides (Saiki et al, PNAS USA 86:6230-6234 (1989)) or oligonucleotide arrays (Maskos et al, Nucl. Acids Res. 21:2269-2270 (1993)), PCR (Newton et al, Nucl. Acids Res. 17:2503-25 16 (1989)), mismatch-repair detection (MRD) (Faham et al, Genome Res. 5:474-482 (1995)), denaturing-gradient gel electrophoresis (DGGE) (Fisher et al, PNAS USA 80:1579-1583 (1983)), single-strand-conformation-polymorphism detection (Orita et al, Genomics 5:874-879 (1983)), RNAase cleavage at mismatched base-pairs (Myers et al, Science 230:1242 (1985)), chemical (Cotton et al, PNAS USA 8:4397-4401 (1988)) or enzymatic (Youil et al, PNAS USA 92:87-91 (1995)) cleavage of heteroduplex DNA, methods based on primer extension (Syvanen et al, Genomics 8:684-692 (1990)), genetic bit analysis (GBA) (Nikiforov et al, Nucl. Acids Res. 22:4167-4175 (1994)), the oligonucleotide-ligation assay (OLA) (Landegren et al, Science 241:1077 (1988)), the ligation chain reaction (LCR) (Barrany, PNAS USA 88:189-193 (1991)), gap-LCR (Abravaya et al, Nucl. Acids Res. 23:675-682 (1995)), and radioactive and/or fluorescent DNA sequencing using standard procedures well known in the art. As will be appreciated, variant detection can also be performed using samples of RNA by reverse transcription into cDNA therefrom. Direct sequencing can also be used. As newer detection methods are developed, they too can be used in variant (e.g., SNP) identification. Genomic DNA suitable for use can be obtained from the individual's cells, such as those present in peripheral blood.

The methodology for preparing nucleic acids (e.g., probes and primers) suitable for detection of polymorphisms is well known in the art. For example, probes can be designed so as to include the polymorphic site and encompass a sufficient number of nucleotides to provide a means of distinguishing a variant from the wild type sequence. Any probe or combination of probes capable of detecting any one of the variants described herein is suitable for use in this invention. Examples of suitable probes include those complementary to either the coding or noncoding strand of the DNA. As detection of polymorphisms can be effected using amplification strategies (e.g., PCR amplification), amplification primers, which can be complementary to sequences flanking the polymorphic site, are within the scope of the invention. Production of suitable primers and probes can be carried out in accordance with any one of the many routine methods. In general, suitable probes and primers comprise, preferably at a minimum, an oligomer of at least 16 nucleotides in length, more preferably, 18 nucleotides long.

Certain variants of the invention can also be detected at the protein level using, for example, antibodies specific for the variant versus the wild type gene product. As will be appreciated, antibodies can be raised against various epitopes of the protein. Antibodies suitable for use in the invention can be present in a kit that is suitable for use in screening and assaying for the presence of α₁ₐAR gene variants by an immunoassay through use of an antibody that specifically binds to a variant gene product, Such kits can include ancillary reagents for detecting the binding of the antibody to the variant gene product.

Antibodies of the invention can be raised and used to detect the presence or absence of the wild-type or variant gene products by immunoblotting (Western blotting) or other immunostaining methods. Such antibodies can also be utilized for therapeutic applications where, for example, binding to a variant form of the α₁ₐAR protein modulates activity.

Antibodies can also be used as tools for affinity purification of α₁ₐAR protein. Methods such as immunoprecipitation or column chromatography using immobilized antibodies are well known in the art.

Antibodies, as well as antibody fragments, against the α₁ₐARs of the invention can be prepared by any of several standard methods (e.g., the antibodies can be prepared recombinantly or using conventional monoclonal antibody techniques).

The invention includes synthetic and semi-synthetic antibodies, such terms are intended to cover antibody fragments, isotype switched antibodies, humanized antibodies (mouse-human, human-mouse, and the like), hybrids, antibodies having plural specificities, fully synthetic antibody-like molecules, and the like.

The present invention also includes gene expression systems that allow for the study of the function of the variant gene products. Such analyses are useful in providing insight into disease processes that derive from variant genes. Expression systems refer to DNA sequences containing a desired coding sequence and control sequences in operable linkage, so that hosts transformed with these sequences are capable of producing the encoded proteins. In order to effect transformation, the expression system can be included on a vector.

In general terms, the production of a recombinant form of α₁ₐAR variant gene product of the invention typically involves the following. First, a DNA encoding the protein (or a fusion of the α₁ₐAR protein to an additional sequence (advantageously cleavable under controlled conditions such as treatment with peptidase to give an active protein)) is obtained. If the sequence is uninterrupted by introns it is suitable for expression in any host. If there are introns, expression is obtainable in mammalian or other eukaryotic system capable of processing them. The coding sequence is placed in operable linkage with suitable control sequences in an expression vector. The construct is used to transform a suitable host, and the transformed host is cultured under conditions to effect the production of the recombinant protein. The protein can be isolated from the medium or from the cells and purified as appropriate.

The control sequences, expression vectors, and transformation methods are dependent on the type of host cell used to express the gene. Generally, prokaryotic, yeast, insect, or mammalian cells are useful as hosts. (See generally Maniatis et al. Molecular Cloning: A Laboratory Manual pp. 1.3-1.11, 2.3-2.125, 3.2-3.48, 2-4.64 (Cold Spring Harbor Laboratory, Cold Spring Harbor, N.Y. (1982)); Sambrook et al. Molecular Cloning: A Laboratory Manual pp. 1-54 (Cold Spring Harbor Laboratory, Cold Spring Harbor, N.Y. (1989)); Meth. Enzymology 68: 357-375 (1979); 101: 307-325 (1983); 152: 673-864 (1987) (Academic Press, Orlando, Fla. Pouwells et al. Cloning Vectors: A Laboratory Manual (Elsevier, Amsterdam (1987))).

Identification of the α₁ₐAR genetic variant protein products has therapeutic implications. The invention encompasses pharmacological, protein replacement, antibody therapy, and gene therapy approaches. In the pharmacological approach, drugs that modulate altered α₁ₐAR gene function are sought. In this approach, modulation of variant gene function can be accomplished with agents identifiable using a screen in which modulation is monitored in in vitro systems (simple binding assays (competitive or noncompetitive) can first be used to identify agents capable of interacting with variant gene products). The present invention provides for host cell systems that express variant gene products and are suited for use as primary screening systems. In vivo testing of candidate drugs can be used as a confirmation of activity observed in the in vitro assays. Rational drug design by use of X-ray crystallography, computer-aided molecular modeling (CAMM), quantitative or qualitative structure-activity relationship (QSAR), and similar technologies can further focus drug discovery efforts. Rational design allows prediction of protein or synthetic structures which can interact with and modify protein activity. Such structures may be synthesized chemically or expressed in biological systems. This approach has been reviewed in Capsey et al, Genetically Engineered Human Therapeutic Drugs, Stockton Press, New York (1988). Further, combinatorial libraries can be designed, synthesized and used in screening programs.

The present invention also encompasses the treatment of diseases associated with the α₁ₐAR variants of the invention using, for example, agents identified using screening protocols such as those described above. In order to administer therapeutic agents, it will be appreciated that suitable carriers, excipients, and other agents can be incorporated into the formulations to provide improved transfer, delivery, and the like.

The present invention also relates to the use of polypeptide or protein replacement therapy for those individuals determined to have a disease-associated α₁ₐAR variant gene. Treatment can be performed by replacing the variant gene product with the wild type protein or its functional equivalent in therapeutic amounts. Proteins suitable for use in such therapies can be prepared by any of several conventional procedures. The protein can be produced, for example, recombinantly or chemically using standard techniques.

Protein replacement therapy requires that the protein be administered in an appropriate formulation. The protein can be formulated in conventional ways standard to the art for the administration of protein substances. Delivery can be effected by packaging in lipid-containing vesicles (such as LIPOFECTIN.TM. or other cationic or anionic lipid or certain surfactant proteins) that facilitate incorporation into the cell membrane. The protein formulations can be delivered to affected tissues by different methods depending on the affected tissue.

Gene therapy utilizing recombinant DNA technology to deliver the wild type form of the α₁ₐAR gene into patient cells or vectors that will supply the patient with gene product in vivo is also within the scope of the present invention. In gene therapy of disease associated with α₁ₐAR variant expression, a wild type version of the gene is delivered to affected tissue(s) in a form and amount such that the correct gene is expressed and sufficient quantities of the protein produced to reverse the effects of the variant gene. Current approaches to gene therapy include viral vectors, cell-based delivery systems and delivery agents. Further, ex vivo gene therapy can also be useful. In ex vivo gene therapy, cells (either autologous or otherwise) are transfected with the wild type gene or a portion thereof and implanted or otherwise delivered into the patient. Such cells thereafter express the wild type gene product in vivo (see generally U.S. Pat. No. 5,399,346).

Retroviruses are often considered the preferred vector for somatic gene therapy. They provide high efficiency infection, stable integration and stable expression (Friedman, T. Progress Toward Human Gene Therapy. Science 244:1275 (1989)). The full length α₁ₐAR gene cDNA can be cloned into a retroviral vector driven by its endogenous promoter or from the retroviral LTR. Delivery of the virus can be accomplished by direct implantation of virus directly into the affected tissue. Other delivery systems that can be utilized include adenovirus, adenoassociated virus (AAV), vaccinia virus, bovine papilloma virus or members of the herpes virus group such as Epstein-Barr virus. Viruses can be, and preferably are, replication deficient. Other methods of inserting the wild type gene into the appropriate tissues can also be productive. This includes calcium phosphate, DEAE dextran, electroporation, and protoplast fusion. Liposomes (i.e., LIPOFECTIN.TM.), synthetic cationic lipids and DNA conjugates can also be used.

Certain aspects of the invention can be described in greater detail in the non-limiting Example that follows.

### EXAMPLE

Experimental Details.

Materials. Drugs and reagents were obtained from the following sources: (−)-epinephrine, (−)-norepinephrine, oxymetazoline, phenylephrine, prazosin, 5-methylurapidil, phentolamine (Sigma); ¹²⁵I-(2-β-(4-hydroxyphenyl)-ethylamineomethyl)-tetralone ([¹²⁵I]HEAT), [³H]inositol, [³H]thymidine (NEN Life Science Products); Dulbecco's Modified Eagle Medium (DMEM) and G418 (Life Technologies); and fetal bovine serum (FBS, Hyclone).

SNP Identification. A systematic sequencing strategy was employed to identify SNPs in α₁ₐAR coding region. Genomic DNA was obtained from 281 individuals (562 chromosomes) purposefully inclusive of multiple ethnic populations (Black, Hispanic, Caucasian, American Indian); sources of DNA included the Coriell SNP discovery panel (Coriell Institute, Camden, N.J.; n=90, enriched for minorities) and individuals from hypertension clinics and hospital settings in Los Angeles, Calif. (n=40) and Durham, N.C. (n=151). Five overlapping PCR amplimers (400-500 bp each) were generated from 1.5 kb α₁ₐAR gene (including 5′ and 3′ regions immediately adjacent to 2 exons), followed by direct double-stranded sequencing of PCR products. SNPs were identified from sequence traces using PolyPhred/Phrap (www.phrap.org) (Nickerson et al, Nucl. Acids Res. 25:2745-2751 (1997)). SNP authenticity was confirmed by manually examining each sequence trace identified by Consed (www.genome.washington.edu) (Gordon et al, Genome Res. 8:195-202 (1998)) (3 most stringent matches used only) and by confirming presence of the SNP in both forward and reverse reads. This was followed by confirmation using at least one of the following criteria: RFLP analysis, second independent PCR reaction in the same individual followed by DNA sequencing, and/or the presence of the SNP in n≧5 individuals in the data set.

In Vitro Site-Directed Mutagenesis in the aoclar. Site-directed mutagenesis was utilized to introduce mutations corresponding to each SNP in hemagglutinin-tagged human α₁ₐAR previously placed in the expression vector pcDNA3 (Price et al, J. Biol. Chem. 277:9570-9579 (2002)). Mutagenesis was performed using the QuickChange™ Site-Directed Mutagenesis Kit (Stratagene) as recommended by the manufacture. All mutations were confirmed by DNA sequencing (Duke University DNA Analysis Facility).

Cell Culture and Transfection. Rat-1 fibroblasts were cultured in DMEM supplemented with 10% FBS at 37° C. Cells were transfected with either the WT (reference sequence with all major alleles, GenBank accession number L31774) or mutated α₁ₐAR in pcDNA3 by calcium phosphate precipitation. For stable transfection, clones resistant to G418 (0.8 mg/ml) were isolated and tested for receptor expression. Two different expression levels were chosen for receptor functional investigation. Low receptor expression level was defined a priori as <0.5 pmol/mg total protein, while high-level expression was defined as >1.5 pmol/mg protein.

Transient transfection was also used to express receptors for binding and IP experiments in order to verify consistent behavior using alternative methods of protein expression. Rat-1 fibroblasts were used because they do not express endogenous adrenergic receptors but do display activation dependent coupling to Gq/11 (Lee et al, Biochem. J. 320(Pt. 1):79-86 (1996)).

Membrane Preparation and Radioligand Binding. Rat-1 membrane preparation and ligand binding assay using the α₁AR selective antagonist, [¹²⁵I]HEAT, were performed as previously described (Schwinn et al, J. Pharmacol. Exp. Ther. 272:134-142 (1995)).

Measurement of Intracellular Inositol Phosphate (IP) Production. Rat-1 cells expressing either the WT or mutated α₁ₐAR grown on 12-well plates were labeled with [³H]inositol for 20-24 h with 2.5 μCi/ml in complete DMEM. Measurement of intracellular IP production was performed under serum-free conditions. After labeling, cells were stimulated for 20 min with various concentrations of NE in DMEM containing 20 mM LiCl. In desensitization experiments, cells were pretreated for 10 min with 10 μM NE, quickly rinsed with DMEM once, placed in DMEM with 20 mM LiCl, immediately stimulated by NE addition and then incubated for 20 min. Total inositol phosphates were extracted and separated as described previously (Price et al, J. Biol. Chem. 277:9570-9579 (2002)). For all experiments, membranes were collected for receptor quantititation and cells were counted at the time of assay.

Measurement of receptor surface distribution and internalization. Cells grown in 6-well plates were treated with 10 μM NE or vehicle for 1 h at 37° C. and then fixed with 3.7% formaldehyde for 10 min at room temperature. All procedures after fixing were done at room temperature. After two washes with PBS, cells were treated with a blocking solution containing 5% non-fat dry milk for 30 min. Rat monoclonal anti-HA antibody 3F10-peroxidase (1:1000 dilution, Roche Molecular Biochemicals) or 3F10 (1:1000 dilution, for testing background signal) was added in subsequently and incubated with fixed cells for 30 min. Following one wash with block buffer and twice washes with PBS, cells were incubated with ABTS solution (Boehringer Mannheim) for 1 h. 1 ml of solution was transferred from each well to 48-well plates and O.D. values were read at 405 nm. The specific O.D. value is from total O.D. subtracting by background signal.

[³H]Thymidine Incorporation. Cells planted in 24-well plates at 1×10⁴ cells/well were cultured in complete DMEM for around 2 days with 1 μCi [³H]thymidine included during the last 4 h incubation. Then cells were harvested and [³H]thymidine incorporation was quantified as described previously.

Measurement of cellular total protein. Cells planted in 12-well plates at 5×10⁴ cells/well were cultured in complete DMEM. After washed once with PBS gently, cells were harvested by 250 μl of lysis buffer (1% nonidet P-40 and 0.5% sodium deoxycholate) at 4, 24, 48, 72 h points. 50 μl of samples were used for total protein measurement by BCA protein assay reagent kit (Pierce).

Statistical Analysis. Results are expressed as the mean±SEM, compiled from n replicate experiments each performed in duplicate or triplicate. Statistical significance was analyzed by one-way ANOVA followed by t-test using GraphPad Prism 3.0 (GraphPad Software, San Diego, Calif.), with p<0.05 considered significant.

Results

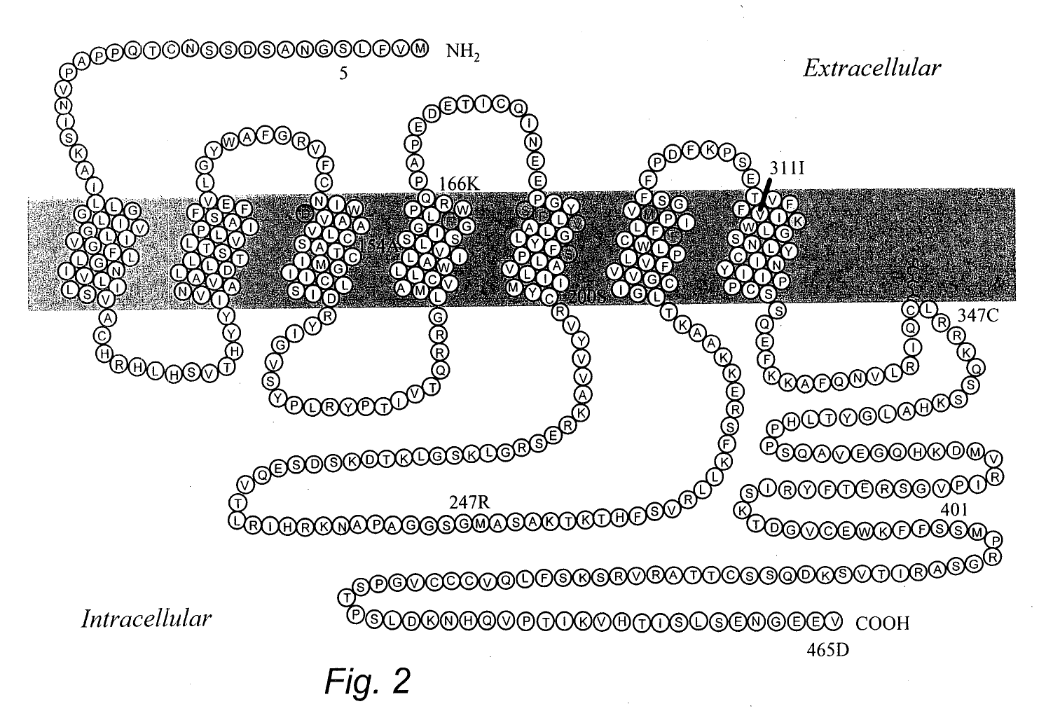

Human α₁ₐAR SNPs. Nine α₁ₐAR coding region SNPs were identified in the present study. Throughout, SNP amino acids are referenced relative to the initiator methionine (M=1), with the amino acid preceding the residue number being WT and following indicating the SNP; nucleotide numbers are relative to the ATG (A=1) in an analogous fashion. Allele frequencies for each SNP for the total population, blacks (n=43), hispanic (n=40), caucasian (n=101) are shown in Table 6. Other groups without enough members to determine allele frequencies were American Indian (n=5) and Asian (n=2) (only R347C was present in these subgroups). As seen in Table 6, SNPs located at nucleotides 15 and 1203, do not induce any amino acid change and therefore were not investigated further. The other 7 SNPs at nucleotides 460, 497, 599, 739, 931, 1039 and 1395 alter encoded residues at amino acid positions 154, 166, 200, 247, 311, 347 and 465 of the human α₁ₐAR protein, respectively. In general, α₁ₐAR coding region SNPs are rare, with 8 having overall minor allele frequency, ƒ(−), from 0.002 to 0.03, except one common SNP, R347C, with ƒ(−)=0.46. FIG. 2 shows the location of each SNP, relative to putative agonist and antagonist binding sites and salt bridge in the human α₁ₐAR.

Pharmacological Characterization. In order to examine whether these polymorphic α₁ₐARs have altered ligand binding characteristics, saturation binding isotherms were first performed to determine the dissociation constant (K_{d}) for the antagonist [¹²⁵I]HEAT, followed by competition assays designed to determine binding affinities for a series of α₁ₐAR agonists and antagonists. Binding measurements with [¹²⁵I]HEAT on membranes from a WT α₁ₐAR high expressing clone indicates a receptor density of 1.77±0.24 pmol/mg protein and a K_{d} value of 42.1±6.5 pM (Table 7). At similar expression levels (1.53−2.37 pmol/mg), receptors containing each SNP display K_{d} values for [¹²⁵I]HEAT not significantly different from WT; these findings suggest alterations of amino acids in these 7 SNPs do not affect the overall receptor [²⁵I]HEAT binding site (Table 7).

Competition binding analysis with the classic α₁ₐAR subtype selective agonist oxymetazoline shows no change in the affinity for receptors containing any SNP (Table 7). In contrast, SNP R166K and V311I, in TM 4 and 7 respectively, causes a significant decrease in receptor affinity for the agonists NE, epinephrine, and phenylephrine (Kᵢ increased ˜3-fold). Experiments using non-radioactive antagonists, demonstrate that the receptor with SNP V311I also has a 3-fold higher affinity for α₁ₐAR subtype selective antagonist 5-methylurapidil (Table 7). In addition, a SNP in TM5 (I200S) decreases receptor binding affinity for the antagonist, phentolamine (Kᵢ increased ˜3-fold). No change in affinity for any variant is noted for the classic non-subtype selective α₁AR antagonist prazosin.

To exclude the possibility that receptor densities or clonal differences might be responsible for altered binding characteristics of receptors containing SNPs, ligand binding was next tested for each receptor from a low expressing clone (receptor densities 0.21-0.44 pmol/mg protein). As shown in Table 8, binding constants obtained are essentially identical to those observed for receptors present in high expressing clones, with exactly the same polymorphic receptors (R166K, V311I, I200S) displaying about the same alterations in agonist and antagonist affinity.

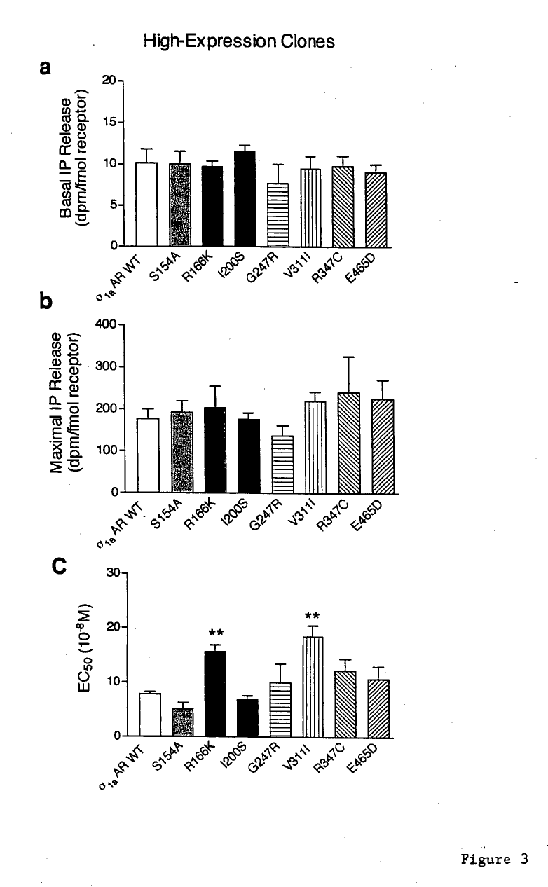

α₁AR Signal Transduction. To investigate whether these SNPs affect receptor activation, each receptor's ability to stimulate IP formation in response to challenge with the endogenous agonist NE was tested. For stable clones at high expression, neither basal IP release (without agonist stimulation, FIG. 3A) nor efficacy of NE (maximal activity, FIG. 3B) is altered with respect to α₁ₐAR WT for receptors with any SNP. However, the same receptors which display decreased affinity for NE, R166K and V311I α₁ₐAR, also display a 2.0-fold and 2.4-fold decrease in potency of NE-stimulated IP formation compared with WT receptor (50% effective concentration [EC₅₀]=0.156±0.012 and 0.185±0.021 μM for R166K and V311I respectively versus 0.077±0.005 μM) (FIG. 3C).

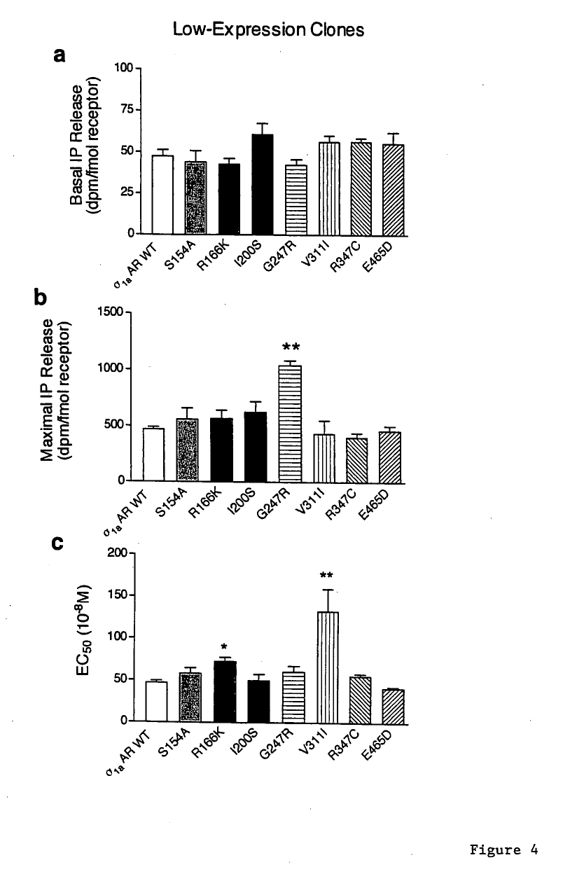

Investigation of IP signaling properties for stable clones at the low-expression level (FIG. 4) show that basal and maximum IP production are the same as the WT receptor with the exception of one receptor (SNP G247R in the third intracellular loop) which displays a significantly higher maximum activity (confirmed in a second independent clone). As observed at high expression level above, the receptor containing SNP V311I or SNP R166K displays a significantly increased EC₅₀ for NE, demonstrating that the decreased agonist binding for this receptor consistently translates into less effective IP production (rightwardly shifted curves).

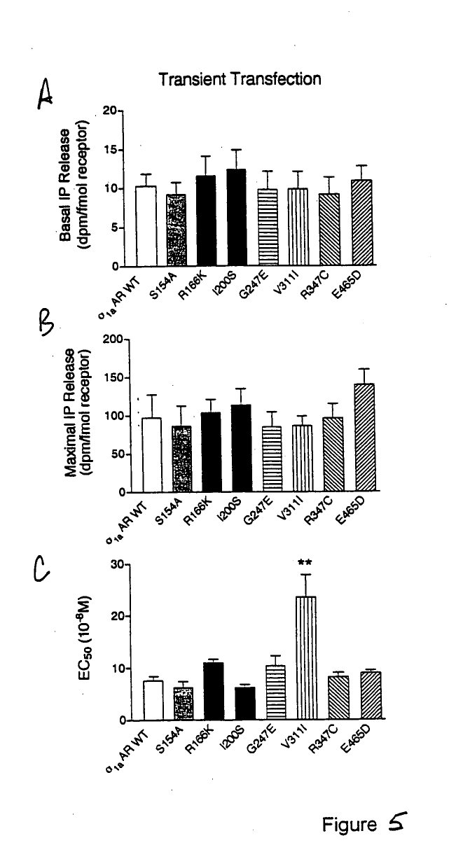

The IP assay was also performed on rat-i fibroblasts transiently expressing α₁ₐAR wild type or its SNPs. Receptor densities were high and fairly consistent for all the transiently expressed receptors (1.30-1.98 pmol/mg protein), indicating that as expected, transient transfection is another high-expression model. Like the high expressing stable clones, IP assays showed no significant change in basal activity (FIG. 5A) or maximum response (FIG. 5B) for any of polymorphic receptors. As in the stable cell lines, receptors with V311I displayed a significantly increased EC₅₀ for NE, demonstrating that the decreased agonist binding for this receptor consistently translates into less effective IP production (rightwardly shifted curves). In contrast, the modestly increased EC₅₀ observed for the receptor with R166K was not statistically significant (FIG. 5C). Although SNP R166K caused decreased NE affinity, this SNP significantly altered IP production only in the high expressing stable clone, suggesting it may not be quite as potent in limiting α₁ₐAR signaling.

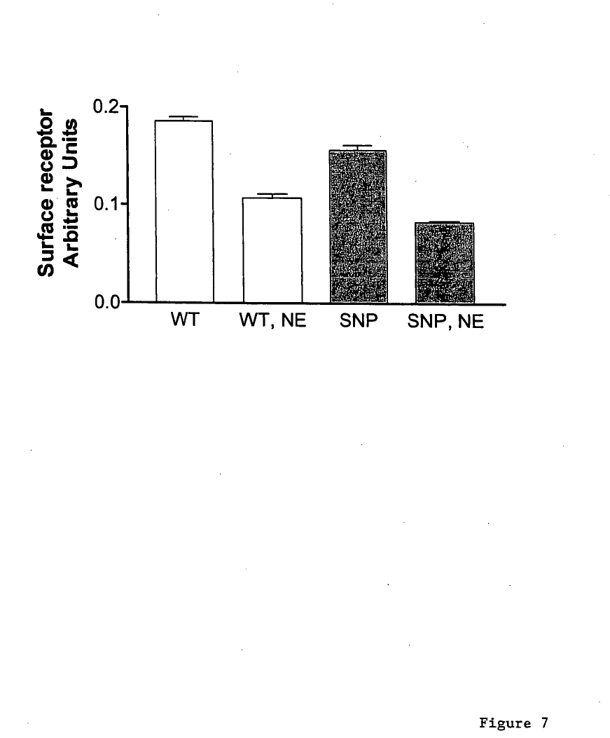

Effects of Human α₁ₐAR SNPs on Norepinephrine-Induced Desensitization. A characteristic of many GPCRs is the tendency of these receptors to elicit less signal with continuing agonist exposure (i.e., to desensitize). Because human α₁ₐARs have been shown to desensitize in response to agonist stimulation (Price et al, J. Biol. Chem. 277:9570-9579 (2002)), the ability of rat-1 fibroblasts stably expressing α₁ₐAR WT or each SNP to respond to a subsequent challenge with NE following an initial NE pretreatment was tested. As expected, compared with unpretreated groups, pretreatment with NE results in 33.5±3.2% and 31.0±3.1% lower IP production in WT α₁ₐAR at high and low-expression level, respectively (Price et al, J. Biol. Chem. 277:9570-9579 (2002)). There is no difference in agonist-induced desensitization between α₁ₐAR WT and receptors with any SNP at either high or low expression level (FIG. 7).

Effects of Human α₁ₐAR SNP G247R on surface receptor expression and Norepinephrine-Mediated Internalization. Since the increased receptor signaling in low-expressing SNP G247R clones is not via a deficiency in agonist-mediated desensitization, the question was raised as to whether the G247R substitution affects receptor distribution (between surface and internal pools) and leads to a redistribution of receptor to the surface or a deficiency in agonist induced receptor internalization. Amino terminus HA epitope-tagged α₁ₐArs were used to address this question. Because anti-HA 3F10 antibodies can only bind to surface receptor after cells are fixed, surface receptors can be measured by the above-described method. As shown in FIG. 7, after normalized by cell count, there is no difference in receptor distribution between WT and SNP G247R cells. The stimulation with NE (10 μM) for 1 h induces both receptors internalization at a similar extent.

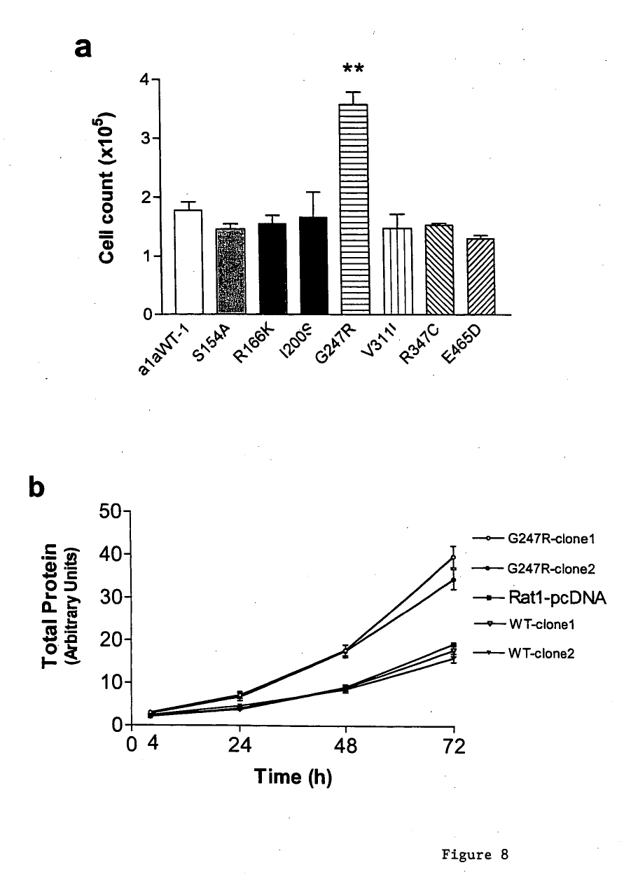

Effects of Human α₁ₐAR SNPs on Cell Growth. Some laboratories have suggested that α₁AR subtypes may modify cell growth. In order to test this hypothesis, equal numbers of rat-1 cells (70,000 cells/well) were plated into 12-well plates for cell counts done side-by-side with IP assays. No difference in cell number is apparent among high-expression stable clones (48 h incubation). But among low expression clones, cells expressing receptor with SNP G247R (2 distinct clones) always grow faster and have a higher (≈2-fold) cell count after incubation for 48 h compared with cells expressing α₁ₐAR WT (FIG. 8A), which was further confirmed by [³H]thymidine incorporation measurement. Since cell growth includes hypertrophy and proliferation, both of which increase cellular total protein, a protein assay was used to further identify whether SNP G247R could induce cell growth. Two low-expression clones for WT receptor, 2 low-expression clones for SNP G247R and rat-1 cells expressing pcDNA vector were plated at same amount cell density, then total protein were collected at 4, 24, 48, 72 h, respectively. As shown in FIG. 8B, SNP G247R clones have higher growth rates than WT clones, and there is no difference in growth rate between WT clones and Rat-1 cells expressing only pcDNA vectors.

Summarizing, seven of 9 naturally occurring SNPs identified in the α₁ₐAR coding region result in amino acid substitutions. These 7 SNPs were investigated for biological behavior in rat-1 fibroblasts through use of high and low expressing stable clones, and 4 SNPs were found to alter ligand binding and/or receptor activation. SNP R166K in TM4 and V311I in TM7 reduce binding affinity for NE, epinephrine, and phenylephrine, an effect which is translated into reduced potency of NE in activating the receptor. Complementing this finding, V311I also increases receptor affinity for antagonist 5-methylurapidil. Quite surprisingly, cells expressing low level of receptor with SNP G247R demonstrate increased efficacy (increased maximal activity) to NE-stimulated IP activity as well as increased proliferative ability. Finally, the receptor containing SNP I200S in TM5 displays a 3-fold loss of affinity for the AR antagonist phentolamine.

Although current knowledge regarding naturally occurring human α₁ₐAR polymorphisms is limited, investigation on mechanisms underlying AR agonist binding and receptor activation has been extensive. Mutagenesis studies suggest that natural agonists, epinephrine and NE, bind to residues in TM3 through TM6 in α₁ₐAR (FIG. 2) (Piascik et al, J. Pharmacol. Exp. Ther. 298:403-410 (2001), Hwa et al, J. Biol. Chem. 270:23189-23195 (1995), Hwa et al, J. Biol. Chem. 271:6322-6327 (1996), Porter et al, J. Biol. Chem. 271:28318-28323 (1996), Waugh et al, J. Biol. Chem. 275:11698-11705 (2000)). In the α₁ₐAR, two phenylalanine residues, F163 in TM4 and F187 in TM5 appear to be involved in agonist-specific binding interactions, as mutation of either of these residues results in a 10-fold decrease in affinity for the endogenous agonist epinephrine (Waugh et al, J. Biol. Chem. 275:11698-11705 (2000)). Interestingly, the mutation in SNP R166K, which causes a consistent reduction in agonist binding affinity, sits immediately above F163, almost one full helical turn earlier in the sequence (3 amino acids or 300° of rotation since there are 3.6 amino acids per helical turn, FIG. 2). The close proximity of this naturally occurring genetic variant to residues that are part of the α₁ₐAR agonist binding site, suggests the R166K change is affecting agonist binding perhaps directly, but more likely through influence on agonist binding residues.

The other SNP which caused a consistent 3-fold decrease in agonist binding affinities, V311I, is located in TM7 near several other residues important in agonist and antagonist binding (Piascik et al, J. Pharmacol. Exp. Ther. 298:403-410 (2001)). This amino acid is located only 2 amino acids carboxy-terminal to K309, a conserved residue in GPCRs, which plays a key role in maintaining the inactive conformation of GPCRs via a salt bridge formed with the highly conserved aspartate in TM3 (D106 of α₁ₐAR) (Porter et al, J. Biol. Chem. 271:28318-28323 (1996)). Upon agonist binding, this aspartate is believed to interact with the amine group of epinephrine or NE (Porter et al, J. Biol. Chem. 271:28318-28323 (1996), Strader et al, Proc. Natl. Acad. Sci. USA 84:4384-4388 (1987), Porter et al, J. Biol. Chem. 274:34535-34538)). Thus agonist binding disrupts the D106 to K309 salt bridge, allowing the receptor to shift into the active conformation. Since bound natural agonists appear not to contact residues of TM7 (Piascik et al, J. Pharmacol. Exp. Ther. 298:403-410 (2001)), the decreased agonist affinity of receptors with the V311I substitution is probably mediated through stabilization of the nearby salt bridge. Further, since V311I is outside the binding cleft on the opposite side of the alpha helix from K309, stabilization of the salt bridge must arise indirectly from shifts in residue conformations important for salt bridge stability. Importantly, the decrease in agonist binding affinity of the receptor with the V311I substitution, consistently translates into decreased potency (increased EC₅₀) for NE induced activation. This probably reflects the fact that the substitution is inhibiting the essential activation step of salt bridge disruption apparently through stabilization of the conformation with an intact bridge.

The V311I substitution also occurs immediately between two phenylalanine residues, F308 and F312, that frequently play a role in antagonist binding for the α₁ₐAR (Waugh et al, J. Biol. Chem. 276:α₁25366-25371 (2001)). Receptors with substitution of these phenylalanines display decreased affinity for select antagonists including prazosin, 5-methylurapidil, WB4101, BMY7378 and niguldipine, but not phentolamine and [¹²⁵I]HEAT (Waugh et al, J. Biol. Chem. 276:α₁25366-25371 (2001)). These changes in binding affinity have been interpreted as alterations in the binding surface for particular inhibitors, thus implicating residues around the V311I substitution as important for antagonist binding to the α₁ₐAR. Thus it could certainly be the case that the binding pocket for 5-methylurapidil is altered by the V311I substitution, resulting in increased affinity for 5-methylurapidil. As with agonist binding, the influence of the V311I substitution must be indirect since this residue is not in the binding cleft (Piascik et al, J. Pharmacol. Exp. Ther. 298:403-410 (2001)).

It is widely recognized that alteration of one amino acid in a receptor may induce changes in conformation, some very subtle, which can influence binding properties of the receptor for particular agonists or antagonists. SNP I200S in TM5 results in a receptor that displays lower affinity for antagonist phentolamine, despite the fact that this substitution is not near the three consecutive residues (Q177, I178, N179) of the second extracellular loop involved in phentolamine binding (Zhao et al, Mol. Pharmacol. 50:1118-1126 (1996)); indeed the I200S substitution is at the opposite end of the TM5 (FIG. 2). Nevertheless, this substitution consistently results in decreased binding affinity in two different expression systems and it is hypothesize that the I200S substitution induces conformational changes that indirectly influence the phentolamine binding site.

One of the most interesting findings is that receptors with a G247R substitution have the same binding characteristics as the α₁ₐAR WT, but nevertheless display increased IP signaling and altered growth behavior at low expression levels (in 2 distinct clones). The absence of significant influence on binding characteristics of this receptor is not too surprising since the G247R substitution is in the center of the third intracellular loop, a nonconserved region that can usually be altered in GPCRs without affecting ligand binding properties (Greasley et al, J. Biol. Chem. 276:46485-46494 (2001)). The α₁ₐAR is fairly unusual for a GPCR containing a full COOH terminus in that the third intracellular loop of α₁ₐAR appears to play the central role in acute receptor desensitization (Price et al, J. Biol. Chem. 277:9570-9579 (2002)). Thus one potential explanation for the increased IP signaling could be a deficiency in agonist-mediated desensitization allowing extended high level IP production. However, under the assay conditions used, no decrease was observed in the ability of receptor with G247R (or any other SNP) to desensitize following agonist exposure. Other hypotheses that could account for increased IP signaling include a redistribution of receptor from internal pools to the membrane surface (Mckenzie et al, J. Pharmacol. Exp. Ther. 294:434-443 (2000), Chalothorn et al, Mol. Pharmacol. 61:1008-1016 (2002)) or improved receptor/G protein-coupling perhaps involving a regulator of G-protein signaling protein. By testing surface receptors, it was found that there is no difference in receptor distribution or agonist induced receptor internalization between WT receptor and receptor with SNP G247R. The finding that receptors with SNP G247R display increased growth is consistent with previous studies suggesting roles for α₁ARs in cell proliferation and hypertrophy (Mimura et al, Biol. Pharm. Bull. 18:1373-1376 (1995), Gao et al, Acta. Pharmacol. Sin. 21:55-59 (2000), Erami et al, Am. J. Physiol. Heart Circ. Physiol. 283:H1577-H1587 (2002), Xiao et al, J. Mol. Cell. Cardiol. 33:779-787 (2001)). Since natural levels of α₁AR expression are generally below even the levels of the low expressing clones, it is likely that low expression clones more accurately reflect natural receptor function.

In terms of clinical significance of the findings, α₁ₐARs play important roles in the pathogenesis of benign prostatic hyperplasia and myocardial hypertrophy, and contribute to blood pressure regulation. Recently, using a gene knockout approach, Rokosh and Simpson (Rokosh and Simpson, Proc. Natl. Acad. Sci. USA 99:9474-9479 (2002)) verified that the α₁ₐAR subtype is a vasopressor in resistance arteries and is required to maintain normal arterial blood pressure. Naturally occurring human α₁ₐAR genetic variants are capable of altering receptor biological activity in ways that can be expected to have clinical implications (see Table 9). For example, SNP R166K and V311I result in decreased binding affinity for endogenous catecholamines and a reduction in the potency of NE. Potential clinical implications arising from these effects (more severe in homozygotes) include protection against sympathetically-mediated hypertension and/or novel mechanism underlying rare human hypotension syndromes. Because the second messenger IP₃ can mobilize intracellular calcium which then mediates vasoconstriction, SNP G247R which produces 2-fold higher IP₃ levels is anticipated to be involved directly in the progression of hypertension. Cell growth is obviously involved in the pathogenesis of prostatic hyperplasia; in addition, proliferation of vascular smooth muscle cells has particular relevance to arterial and venous remodeling in hypertension and atherosclerosis, and possibly in cancers such as prostate cancer (Siu et al, Prostate 52:106-122 (2002)). The proliferative effect of SNP G247R is indicative of a role in these diseases.

All documents and other information sources cited above are hereby incorporated in their entirety by reference.

What is claimed is:

1. The method of detecting disease in a patient comprising screening DNA present in a sample from said patient for at least one mutation in the α₁ₐadrenergic receptor (α₁ₐAR) gene, the presence of said mutation being indicative of disease or predisposition to disease.

2. The method according to claim 1 wherein said disease is a cardiovascular disease, a psychiatric disease, or cancer.

3. The method according to claim 2 wherein said disease is hypertension, atherosclerosis, or myocardial hypertrophy.

4. The method according to claim 1 wherein said disease is benign, prostatic hypertrophy.

5. The method according to claim 1 wherein said mutation is a point mutation.

6. The method according to claim 5 wherein said point mutation results in an amino acid substitution in the encoded α₁ₐAR.

7. The method according to claim 6 wherein said point mutation results in the substitution of arginine for glycine²⁴⁷.

8. A method of detecting the presence of disease in a patient comprising:
i) obtaining a biological sample from said patient; and
ii) screening said sample for a mutant α₁ₐAR,
the presence in the sample of said mutant α₁ₐAR being indicative of the presence of disease or predisposition to disease.

9. The method according to claim 8 wherein the sample is a biological fluid or tissue sample.

10. The method according to claim 9 wherein said sample is a biological fluid and said fluid is plasma, serum, urine, lung lavage, ascites fluid, saliva or cerebrospinal fluid.

11. The method according to claim 9 wherein said sample is a tissue sample.

12. The method according to claim 8 wherein said screening is effected by contacting said sample with a compound that forms a complex with said mutant α₁ₐAR under conditions such that the complex can form, and determining whether any such complex forms.

13. The method according to claim 12 wherein said compound is a binding protein.

14. The method according to claim 13 wherein said binding protein is an antibody or binding fragment thereof.

15. The method according to claim 8 wherein said disease is a cardiovascular disease, a psychiatric disease, or cancer.

16. The method according to claim 15 wherein said disease is hypertension, atherosclerosis, or myocardial hypertrophy.

17. The method according to claim 8 wherein said disease is benign, prostatic hypertrophy.

18. An isolated antibody specific for a mutant α₁ₐAR.

19. The antibody according to claim 18 wherein said antibody is a monoclonal antibody.

20. A kit for use in the detection of a mutant α₁ₐAR comprising a compound that specifically binds to said mutant α₁ₐAR disposed within a container means.

21. A method of detecting disease in a patient comprising contacting a biological sample from said patient with at least one mutant α₁ₐAR under conditions such that said mutant α₁ₐAR can bind to autoantibodies thereto present in said sample to form a complex, and detecting the presence of said complex,
wherein the presence of said complex is indicative of disease or predisposition to disease.

22. The method according to claim 21 wherein said disease is a cardiovascular disease, a psychiatric disease, or cancer.

23. The method according to claim 22 wherein said disease is hypertension, atherosclerosis, or myocardial hypertrophy.

24. The method according to claim 21 wherein said disease is benign, prostatic hypertrophy.


SEQUENCE LISTING (44 sequences)

SEQ ID NO: 1 - protein, 466 aa, Homo sapiens [sequence omitted]

SEQ ID NO: 2 - DNA, 12 bp, Homo sapiens
caatcagatttg

SEQ ID NO: 3 - DNA, 12 bp, Homo sapiens
tttgtcatattt

SEQ ID NO: 4 - DNA, 12 bp, Homo sapiens
tatttaaaacac

SEQ ID NO: 5 - DNA, 11 bp, Homo sapiens
tacaaatgact

SEQ ID NO: 6 - DNA, 12 bp, Homo sapiens
gaggaaaacatt

SEQ ID NO: 7 - DNA, 11 bp, Homo sapiens
tcagcgagagc

SEQ ID NO: 8 - DNA, 13 bp, Homo sapiens
tttttaaaaaatg

SEQ ID NO: 9 - DNA, 14 bp, Homo sapiens
gacatcagtggtgg

SEQ ID NO: 10 - DNA, 13 bp, Homo sapiens
ggggtgagtcagc

SEQ ID NO: 11 - DNA, 11 bp, Homo sapiens
ggtccgcatcc

SEQ ID NO: 12 - DNA, 13 bp, Homo sapiens
cctcccccgccgt

SEQ ID NO: 13 - DNA, 12 bp, Homo sapiens
ataccggcccct

SEQ ID NO: 14 - DNA, 11 bp, Homo sapiens
cctgccgagtc

SEQ ID NO: 15 - DNA, 11 bp, Homo sapiens
ctgtcgcttgc

SEQ ID NO: 16 - DNA, 13 bp, Homo sapiens
gaaaggcgtcatg

SEQ ID NO: 17 - DNA, 12 bp, Homo sapiens
ggcgtcatggac

SEQ ID NO: 18 - DNA, 13 bp, Homo sapiens
tggctctcaagcc

SEQ ID NO: 19 - DNA, 12 bp, Homo sapiens
gccagcttggct

SEQ ID NO: 20 - DNA, 11 bp, Homo sapiens
gagaatgagcg

SEQ ID NO: 21 - DNA, 11 bp, Homo sapiens
ctcaaaatgta

SEQ ID NO: 22 - DNA, 10 bp, Homo sapiens
ggaattgcat

SEQ ID NO: 23 - DNA, 12 bp, Homo sapiens
tttaatgccctg

SEQ ID NO: 24 - DNA, 14 bp, Homo sapiens
agcccgggaggtgg

SEQ ID NO: 25 - DNA, 13 bp, Homo sapiens
ctctcgggaaatg

SEQ ID NO: 26 - DNA, 13 bp, Homo sapiens
cactctccctggt

SEQ ID NO: 27 - DNA, 14 bp, Homo sapiens
ggctggaggcagcc

SEQ ID NO: 28 - DNA, 10 bp, Homo sapiens
ccatcatcct

SEQ ID NO: 29 - DNA, 14 bp, Homo sapiens
ggcagcgggatggc

SEQ ID NO: 30 - DNA, 12 bp, Homo sapiens
cctcggcttctg

SEQ ID NO: 31 - DNA, 14 bp, Homo sapiens
tttcaaacagcaca

SEQ ID NO: 32 - DNA, 13 bp, Homo sapiens
tccagtctcattt

SEQ ID NO: 33 - DNA, 10 bp, Homo sapiens
tctccgcaga

SEQ ID NO: 34 - DNA, 12 bp, Homo sapiens
ttctcttccatg

SEQ ID NO: 35 - DNA, 11 bp, Homo sapiens
ggtacccaccc

SEQ ID NO: 36 - DNA, 11 bp, Homo sapiens
ttctcggaagg

SEQ ID NO: 37 - DNA, 12 bp, Homo sapiens
atgcagaagggg

SEQ ID NO: 38 - DNA, 7902 bp, Homo sapiens
  misc_feature (7608)..(7608): n is a, c, g, or t
taaaccatgttttggtataaatgtaataatagaatgagttcatgatataatataagtgaaaagtaagatatataattataggtacagtatgattacactcttttatataatgtatacaatgtataatatgtgaaatatatatgtatgcataataaatacatgtacataaaatatttcctggtatgtacatgtgtatatatatttttccatgtatataattaatacacattaacaaaaaatgagaaaatatgacaaatatttaacaatagctatttctccagtggcaggattacagataatttttattttcatacatttctgtctgacttttccaaatgtgctattatgagcataaattttataattaaatacaagtcaataagcattttaatgtgcctataacagcccatcagcaagtgaagagatgagataaaaatgtgtctgcacttggtacacatattgactttcttttccttccatttccagatgatattgacaaagatatttggggggttgattaatatgcctcagcttagatttgctatgcgagagcaaagtcttattgttttaaaggaattatttgctgatcagctataggctgacttgacctcatattcctaccatgatagtcccacagtgtagggatggggtggtatgactcccaagacttggagtatgttattttctcagggcaatgaagatttggaaaaatgatggcaaagacagaattactatctcatcaaagacatccctcagcagtatctgtgggctgtgcagtgttctcagttcgataaaggattagaacacaatgcttctttggagagctgtgacttgatactgcatcaatacctttctgagaattgttttttcattttcttgcctctttaacttcttaagccttaggagaattagttgaaaagccaagtctttggggtagatactaacattaagtcttctactctgtcatttgcaatcataaattccagaacacagctcctaattccattgtgtattgttttctaagggaatgatagacagattctttatttttttaaacctctaagcctaccacacttgccgagttcctcactagtcactaagaaagtcctgccaatcaatgcatgggtttatgtccattgctcagctcttctccaatcagactcattcccccagcatccctgacacaccactctaaaatgcggctgctgatggttcaccttcctcacttttgtctacaaatctcaatcctgctgattccacaaatcctacatcaagcaatatcattttatgagtctttccacaaccaccccttcaggggattcttcaatttctgtcacaccggaagtcttcagagtatcaccctcagagccaggcaagagggaccccggctagggtttcaggctttagagagtccagctctgactccttttggccataggactaatgtgatatgcccacctggagcctgtgccctcctttctagaccatgccctgggactcagaatcccttgccccagatggccacacaatcactttcaggtccattctctctgggcagacaacatcacaaatgtgtgtaccccaaggcctgaggccaagaaggcagctttctggctgtaggggctgaggtgttcacacacatttgcatggcccctcaagacaaagaacaagggggaaagtgagaagaaaagaagcagccagtgatcagggccagctcttgcaacttagccatgttgggtcattctgattaaaccacttagctcaagtgtagtgctcaagacacttagcacattctccagctgaatttaccagtgttcatggacgacctgggttagaaatatatttcgctataaagtagcatacaaaatgagcagaaagggagttaataagattaataatagagttagtgaatattatgagctgagtttttgagaaacgtaatttctttcacaacactaataacaaccttgtgggggttcattgtctccctttaaaaattaggaaaccaaggctttgccatggtcgcataggagggtcagaatagcatctttatgacccagagcatactcgtctccactccacctacccatgtgtacaactcagacactttctgggatgtccacgtcaactattctttaaagagtaaccaacagatggatagttttctgtttgtgaatcaatggtaggtgactgaaaaattggttctgagaggtcgttttgcaaggattgatggtcacaggctgagaagcagatttgaaagacctacctgctagcagcataagagctgctcttccttatcttagtattaactagttaattattggaggtgggtgcaggggtggattatgtgtattcttaattgttgtagagtgggaactgggagttacaaagacttttgcaagtttcgaccttgcagagctgagcaattttcagttgctttgcttgctgatagcactgcttcccttatctaccatggaacacatcttaatgaagaatttgcattcacagcatcaggttaatgaatacaaaacaaaacagtgtatatccctctgatggatgggatttcggaagcacagacattatacacatatttgatgataaagtactagaagtgcagggaattgaggtcaagcttcctcctaaggggactgaatcccagagagagcaggtgacttagtaatgagaagtggagctgtctgttcaaccaggatgctcctcctatggcacgaaattcagttttaaaaatatattaaattcaaatcaaatgtgttaggtgtgagttcttatccctacaggtatgaggcagaggtggaggactttgtatacaatagagaaataaatacatatattaggtcttccatgacataggatttactgaccctctcatgggcattcctctgaggcattttgagatttattgctataaaagagcctcccaaacattatctcacttagaaaaggtaatcatattaatatgattttgttcacaggagagaatttaagtgccactgcttaaagttatctccttgttcctaggtttaaggagacctagtaaataagaacattccactttgtctgcatcaataaagatgaaagatgacttaggaggtgggaattggagtgggaaacatttttctatgttcccgatattctgaaacacatgtgactttattcaatcacaaggtaaacagattatgtaatttaccagaaaaaaagtaataagactggtggtgctaggttttcatactccagctattaatgaattaaagagagtaacactcctgaaaggataccattttctcaagaaaactggaaaagattgtgtggcatttaaaaaataccaaactctgtggccataatgctcttaaaattcatctgtctaaagaaattagaagtgaatcatattaaataaggtttagatatgtccactttatcttcctgaaaatataatttcattacaatcagatttgtcatattttatctgattttacttgctatttaaaacaccttataatttacttgcatatttagaattacaatattcttaatatacttcttgatcttaacaaaacctaggccaaatgttaatcaaatcaagctgttcaaagttactttatagcacattcctatgaacacaccatacacacagcaatatctagcaagggtgtcaatttttcgttatttttaaaagctcatttaaagaagttatttactacaaatgactctacacacacacacacgcgcgcgcgcgcgcacacacacacacacacacacacaaacctttttaaagaaacgctagaacccaaccccctctaggccagaggaaaacattacagctgtatacgcacttgtgcctgttgccgtagagtaatacggtagcagcaggagattacggtactagctgggctactgcctgagttacgtcagcgagagctgcaaagttccttgctattcttttctggtgtcggggagctgaatattaaaagggtgattgtggagttaccggttatctgcatttttttttcttttcttattttgactctttttaaaaaatgcaggtaaagtgacagcggttcaggagcttaaagacatcagtggtggaggggtgagtcagcgggtgcaaaaggacaaggatttggtgcctcggagacacggtcccctctccgcctccagagaagagcaggcaggcagctcccgggaccgaagccgggtccgcatcccccgcgcgcgagctggtggctcagcagcggcgcttcaggtgagtgcgccggggccggcgtcccgcagggccgagtgggtgagggcagacctcccccgccgtctggtgagacggaacccccacttttcccagcgcctcccgctttttccaccaggttttataccggcccctctaccccacccccgattcccttacatcttctgcgaagttgccttctactgaacaagtgtctttttaaccctgtgtttatcaccctcgaggtaggaggaaaagggtttctgcagtggcacgtttttaataccacctgtgaggtctccaacttgcgattttaacaagagtctttgcccgaggtcccacctcagggcccaaccccagaaggcaaggtgggcacttcctcacgccgcgctgtcctgccgagtccctgcggtaggttcgcagttgtggaaacccaggtttcttacgcagatggtggcccccagcccagaaaatcgaaggcggcccctgcccgctggcatgccggcttaatgtttacgcctgcaaaatccgcagtgactgtcgcttgcaaagctccctctgcagagggacgtcctccccaccccgtcccccgccagtcccgctacggctggcagctggagcccctcgggtggccaacagtgaggcttggaaaggcgtcatggacagacctgggtcgctttctgtcttcgggtccctcccggcttcgctcgggacctggctctcaagccagcttggctggtggacagaccggtgcgctctgcacacccgagtgcgaattccaccggcgtgagaatgagcgtgctcgtggtcctggccctgaggtccctgggtcgcagctgttccctctcccaggccgccccctccaggtgactgcgaggcaacctgttctaacggaaaccgagtacatcctccagaattccccggctaggatccgtgcgacacactcgccagccgcagtcgcccctccggggcttcgaggattttaatttcgtggtacctgcgctcgaaatccagacttcgagcgctggagcctggggttttggggatttgtttttttgtttgtttttcgcttcggatcctgaactcgggcagaggtgactcagtagagtgcgctaggcaggttcccagtggtgggggcgcgagatgagctccgaagtcgcctccaccgctgccgggcgaagcagcttctggaccgcagaaccaacccggctcccaactggtgtcccccaacccgtcaagctcagcacagcctctttccctggggcgcctagctcaaagccgcctttctctttgcgctctttcaggtggacgcggtcaaacgatgccccgcagcctcctgggtctcagcacatattccacacctacgtcccctgacctgtgctcctagaagctggagagagcaggagccttcggtggggcagctcaaaatgtaggtaactgcgggccaggagcagcgcccagatgccatcggtccctgcctttgagcgtcgacggctgatcttttggtttgagggagagactggcgctggagttttgaattccgaatcatgtgcagaatgctgaatcttcccccagccaggacgaataagacagcgcggaaaagcagattctcgtaattctggaattgcatgttgcaaggagtctcctggatcttcgcacccagcttcgggtagggagggagtccgggtcccgggctaggccagcccggcaggtggagagggtccccggcagccccgcgcgcccctggccatgtctttaatgccctgccccttcatgtggccttctgagggttcccagggctggccagggttgtttcccacccgcgcgcgcgctctcacccccagccaaacccacctggcagggctccctccagccgagaccttttgattcccggctcccgcgctcccgcctccgcgccagcccgggaggtggccctggacagccggacctcgcccggccccggctgggaccatggtgtttctctcgggaaatgcttccgacagctccaactgcacccaaccgccggcaccggtgaacatttccaaggccattctgctcggggtgatcttggggggcctcattcttttcggggtgctgggtaacatcctagtgatcctctccgtagcctgtcaccgacacctgcactcagtcacgcactactacatcgtcaacctggcggtggccgacctcctgctcacctccacggtgctgcccttctccgccatcttcgaggtcctaggctactgggccttcggcagggtcttctgcaacatctgggcggcagtggatgtgctgtgctgcaccgcgtccatcatgggcctctgcatcatctccatcgaccgctacatcggcgtgagctacccgctgcgctacccaaccatcgtcacccagaggaggggtctcatggctctgctctgcgtctgggcactctccctggtcatatccattggacccctgttcggctggaggcagccggcccccgaggacgagaccatctgccagatcaacgaggagccgggctacgtgctcttctcagcgctgggctccttctacctgcctctggccatcatcctggtcatgtactgccgcgtctacgtggtggccaagagggagagccggggcctcaagtctggcctcaagaccgacaagtcggactcggagcaagtgacgctccgcatccatcggaaaaacgccccggcaggaggcagcgggatggccagcgccaagaccaagacgcacttctcagtgaggctcctcaagttctcccgggagaagaaagcggccaaaacgctgggcatcgtggtcggctgcttcgtcctctgctggctgccttttttcttagtcatgcccattggtaagtcttgaacacccctcactttagcatctgggggtcttcaccctcctcggcttctgttaccccagactcccagtccgggatggaagaggaaggattagcatttcaaacagcacagctctagggcaattagaaaaggctcccttgtagaaaagtgaattttcattctctttctactccagtctcatttatattaggtcctagagcactttttcgactgtaaagtggcttccaactgatgcagattaattggtctctttaataagaatgtcaacttttcttaatgcctataagcacgtgttcaatttaaatgcatctgctctctctagtctcagagtctccaccaagtgcttaggctgactgtggaatgccattttcactctgctacagaatgcaaattctcttggcctgaaaataagtaccatgcttattctggacaaatgtgtgattttattattgcattaggttattcataagggtttgttataatggtctgtttatgttctatatctgtgctaantttattttctggattcagtatggaaggaattatggtcagccactnagaaaaaaaaatgattttatgtccaaaccaatttaagccttaaataattaatcatagtatttccaataagtaaatacttatttttttattttaataataagtattaaaaacaaacactttctcttatccaaaaatcatccgggaaagttacaagataacactgtttgaaaattatacagtacncataatgttacaaatccaatttttgcaaatgctaaattngcgtttgtcaaaattaaattgctcatc

SEQ ID NO: 39 - DNA, 1915 bp, Homo sapiens
  CDS (456)..(971)
  misc_feature (1496)..(1497): n is a, c, g, or t
ataaacactgaggctgtgtctgttgcataacactgcatcagagaataaaaggcatgttcagataaccgaattttaatatggattacttgcatggattccaacttacttttcaatttaggcaaaacaatttacatatgtggactcagtctgagtttcacattttcatttggtaaaacttcacagcagctgttggtcactgagagccagtgcaaccctacccactgggcctgctcctgtaattaatgacacacgcggaccaagtaggaatggtctttgaagatattgcaaaagggtgacagtcataggagctagtcagtcaaatgtgagaaactcatatgtgtttgggatcattttaaccgtttaaaaatacagaaagatgtctgtttgattgttttcctagccaattggcttgctggctttcaaataatatgtataaatctgtgtgttttcttccagggtctttcttccctgatttcaagccctctgaaacagtttttaaaatagtattttggctcggatatctaaacagctgcatcaaccccatcatatacccatgctccagccaagagttcaaaaaggcctttcagaatgtcttgagaatccagtgtctccgcagaaagcagtcttccaaacatgccctgggctacaccctgcacccgcccagccaggccgtggaagggcaacacaaggacatggtgcgcatccccgtgggatcaagagagaccttctacaggatctccaagacggatggcgtttgtgaatggaaatttttctcttccatgccccgtggatctgccaggattacagtgtccaaagaccaatcctcctgtaccacagcccgggtgagaagtaaaagctttttgcaggtctgctgctgtgtagggccctcaacccccagccttgacaagaaccatcaagttccaaccattaaggtccacaccatctccctcagtgagaacggggaggaagtctaggacaggaaagatgcagaggaaaggggaataatcttaggtacccaccccacttccttctcggaaggccagctcttcttggaggacaagacaggaccaatcaaagaggggacctgctgggaatggggtgggtggtagacccaactcatcaggcagcgggtagggcacagggaagagggagggtgtctcacaaccaaccagttcagaatgatacggaacagcatttccctgcagctaatgctttcttggtcactctgtgcccacttcaacgaaaaccaccatgggaaacagaatttcatgcacaatccaaaagactataaatataggattatgatttcatcatgaatattttgagcacacactctaagtttggagctatttcttgatggaagtgaggggattttattttcaggctgttcacttactgcacagccatttcaacatggcttacaaaagcctttcttgacaaatcacttacctgttccagaactctgttatgagaatccagagcttataatattttgnnaggcaaaagattgtctcccattacttcttatctgcttcactattgcataatgaatgagcttcacctgtggcatgttggaatgagccttatgatccaagtacattattcccgaactttgnaaaatactaatgcttagcttcagacaatactgatggntcnccaaagnactgtctaacgcaggagtttncnaacattttttgataggaggccatttgttctggtaaaagatccagtagtcaactcaacttcatctattcctacttttctgcaagagcttggggnacatgctatattttgctttatgtataccnaattttggtaaaccataataactcagtaaaaaaaaagtcgacgcggccgcgaattcgatatcaagcttatcgataccgtcgacctcgagggggggcccggtacccaa

SEQ ID NO: 40 - protein, 172 aa, Homo sapiens
GSFFPDFKPSETVFKIVFWLGYLNSCINPIIYPCSSQEFKKAFQNVLRIQCLRRKQSSKHALGYTLHPPSQAVEGQHKDMVRIPVGSRETFYRISKTDGVCEWKFFSSMPRGSARITVSKDQSSCTTARVRSKSFLQVCCCVGPSTPSLDKNHQVPTIKVHTISLSENGEEV

SEQ ID NO: 41 - DNA, 1702 bp, Homo sapiens
  CDS (765)..(860)
  misc_feature (6)..(6): n is a, c, g, or t
aagacnagttgtgttttgggtaaatnaaanantctcntnnngatttttgntnaggccctacagtntgccaggnatcttccagganttttgaacccattgcctctaaantcnttggaaactcttgtacccctattttaacaganaaaaaatctgatcattgtanggagnttaaggacttgcccaaggccaaggggacccatgatttaaacctggtctccctattctcaacntgcacattttccatagcccccccttcctcaagagaatgggggtaaacgttttcccatttggattaggtgtgctgaggatgccaagccatatccaaactttttaatgttctgtttccttgagatttgcctccaaattaaccatggcaaacatgctgccaaatctccagcccagtcaaacccaagagcagggcatctgtggaagaatttggtgtctgcacttctatcagactgcatcacatttttaggagttaatggcctggaatgtgattaaggccttgcaaggaggacatactatggcacgctgggggaagatgggcatagaagagtatgcagaaggggccacattggccaagaacagtaaaatgcagttgctgacaggacacatatcgggtgttgtattgaagttattgatgaccaaccacagttcatagaaacacttttgggaagtacatcccttttaaaaataaatgaaagcaaatacggcataactcactctcactcacctgtattccaactttttttttgtttggacagaggggaatggattgtagatatttcaccaagaattgcagagagcatatcaagcatgtgaattttatgatgccaccgtggagaaagggttcagaatgctgatctccaggtagctggagacctaggcagtctgcaaatgaggagtcagctggaagctatggctatgtattatgtgacatcgcttgttcctaatttcttttcacacaagtgaaaactggatatcccaaccttctggcccagtaggtttcatggttaagacctggtagtgagaacattttaggaactatttgcttgggcaggtaatttttcactctgatcacagctacttaatcagagcttgacaaactttctcaattgcttctggggctggtctgctcaggtcctttggccaaaaaagatgctgcctctgtgtgtgaatacttgttgacttaattaaagaaagagctctgctcattagcaaagggcactgnngcagatgggaggtaaactctccagggaaaaaccaagtgaaaagaaagcagaggaggcaaatatggagacatcagaggtatgcctaccagttactctgatttttttacactactaggacttttaactatgaaaccacctgcgcacagctccagtggagcccagttggaacactgtttgcacctgccccgtatctgcagactgtcctggggagctgggctgagccaggctgctgtggtgccatccatcttcagaaagcaatggcagctgtggccctcctggcctccanaacccctggggagcaagnatgagtgggagatnatcactggtgggggctnaggctgacnaaaagcaagtttaggaatttcaatngggggggacaacaagccctngcncccatcnagcaattaggtcaattcacgccnccccaagacccaaatntgggnggagggggttnnagantttggcccttc

SEQ ID NO: 42 - protein, 32 aa, Homo sapiens
RGMDCRYFTKNCREHIKHVNFMMPPWRKGSEC

SEQ ID NO: 43 - DNA, 1214 bp, Homo sapiens
  CDS (831)..(848)
  misc_feature (11)..(11): n is a, c, g, or t
ccctcattgcnaacanttgaagcatnttnaaggngtggcntnctttttgtaaccaaaacctgattggaagcagaagttggggggggggggaaattggaagcaaaagggaccgaacttgaagcntgtactncccagacttctcattggaagctccaggtcacaatcttagtnctaatttcaggttcctgcccagtcgagtctagacattnctggggcaccctttaagtggtctccagcacccttctaggcaggtggcttcacagggaagtccacactgcaagctcagctcaccacgactcaggctgacggggtagtcagccatgctcggaggctgaatttggcagggacttgctgccatctccctgccaaatggtctctcgtgactcagaatctcaaacttgttttaaagagaggaaaaaagtcactttcggggatgaggttcttggcccaactctgctttatataaacacagtctatggctatttcagtcttctggattttgagaagcagctgcaaggatgaacggattggtgttggcccaaattaaaaagaagagtattcagttctttcagtgtttggagaaagaagaccaaaagcatcatctcacagggagcagaatgtgaccagcctggctaatgaggaaatgagagggatcctcaacttgagaacccgctctactgaagtctgaacttggaaaaatggacacattgggtttggagtaagaattcttactctacaaaaggataaaattgtgatcacattgatgcatgatgcctaggatattaaaaatgcatgattaattaaatgttagtctaccttgtgttttaaagggacacacacccatgacatgaagccagcttcccgtccacgactgttgtccttactgcccaaggaaggggagcatgaaacccaccactggtcctgcgacccactgtctttggaatccaccccaggagcccaggagccttgcctgacacttggatttacttctttatcaagcatccatctgactaaggcacaaatccaacatgttactgttactgatacaggaaaaacagtaacttaaggaatgatcatgaatgcaaagggaaagaggaaaagagccttcagggacaaatagctcgattttttgtaaatcagtttcatacaacctccctcccccatttcattcttaaaagttaattgagaatcatcagccacgtgtagggtgtgag

SEQ ID NO: 44 - protein, 6 aa, Homo sapiens
GHTPMT